(19) United States
(12) Patent Application Publication
(10) Pub. No.: US 2024/0416097 A1
(43) Pub. Date: Dec. 19, 2024

(54) MICRONEEDLE INJECTOR AND METHODS OF MAKING AND USING SAME

(71) Applicant: Merck Sharp & Dohme LLC, Rahway, NJ (US)

(72) Inventors: Daniel A. Bonanno, Rahway, NJ (US); John B. Cline, Rahway, NJ (US); Eduardo Cristofolli, Rahway, NJ (US); Cassie Megna, Rahway, NJ (US); Wail A. Rasheed, Rahway, NJ (US); Ophelia L. Wells, Easton, PA (US)

(73) Assignee: Merck Sharp & Dohme LLC, Rahway, NJ (US)

(21) Appl. No.: 18/701,321

(22) PCT Filed: Nov. 1, 2022
(86) PCT No.: PCT/US2022/048568

§ 371 Date: Apr. 15, 2024

Related U.S. Application Data
(60) Provisional application No. 63/275,096, filed on Nov. 3, 2021.

(51) Int. Cl. A61M 37/00

(52) U.S. Cl. CPC A61M 37/0015; A61M 2037/0023; A61M 2037/003; A61M 2037/0061

(57) ABSTRACT

In some examples, an injector includes a base, a housing movable relative to the base, a needle unit disposed within the base, the needle unit comprising at least one piercing needle configured to pierce a pierceable membrane of a drug product container, and at least one microneedle disposed on an opposite side of the at least one piercing needle, and a driving mechanism configured and arranged to drive at least one of the drug product container and the needle unit toward another of the drug product container and the needle unit.

### FIELD OF THE DISCLOSURE

The present disclosure relates generally to drug injectors. More specifically, the present disclosure relates to microneedle injectors that facilitate the administration of drug products.

### BACKGROUND OF THE DISCLOSURE

Pre-filled syringes are single-dose devices that contain vaccines, biologics, medicaments, and the like to which a needle has been fixed by the manufacturer. Such syringes typically utilize parenteral routes (e.g., injecting directly into the body, bypassing the skin and mucous membranes), and are administered by a physician or healthcare provider. Recently, automatic injection devices (commonly known as “autoinjectors”) have been used to simplify the administration of drugs in certain settings. Due to their simplicity of design, autoinjectors allow patients to use the devices on themselves reliably and safely, and in their own home without supervision.

Certain patients may experience pain while using conventional autoinjectors, especially in multi-dosage treatments, and recurring therapeutic applications. Additionally, conventional devices may not be able to allow for proper sustained drug release.

Thus, there exists a need for devices that improve upon and advance the methods of delivering drugs and medicaments to patients in a safe, comfortable, and controlled manner.

### SUMMARY OF THE DISCLOSURE

In some embodiments, an injector includes a base, a housing movable relative to the base, a needle unit disposed within the base, the needle unit comprising at least one piercing needle configured to pierce a pierceable membrane of a drug product container, and at least one microneedle disposed on an opposite side of the at least one piercing needle, and a driving mechanism configured and arranged to drive at least one of the drug product container, and the needle unit toward another of the drug product container and the needle unit.

In some embodiments, a system for drug delivery includes a drug product container having a pierceable membrane, a needle unit disposed adjacent the drug product container, the needle unit comprising at least one piercing needle configured to pierce the pierceable membrane of the drug product container, and at least one microneedle disposed on an opposite side of the at least one piercing needle, and a driving mechanism configured and arranged to drive at least one of the drug product container and the needle unit toward another of the drug product container and the needle unit.

In some embodiments, a method of delivering a drug product includes providing a drug product container having a pierceable membrane, providing an injector including a housing, a base moveable relative to the housing, a needle unit disposed adjacent the drug product container, the needle unit comprising at least one piercing needle and at least one microneedle disposed on an opposite side of the at least one piercing needle, and a driving mechanism configured and arranged to drive at least one of the drug product container and the needle unit toward another of the drug product container and the needle unit.

### BRIEF DESCRIPTION OF THE DISCLOSURE

Various embodiments of the presently disclosed microneedle injectors are disclosed herein with reference to the drawings, wherein:

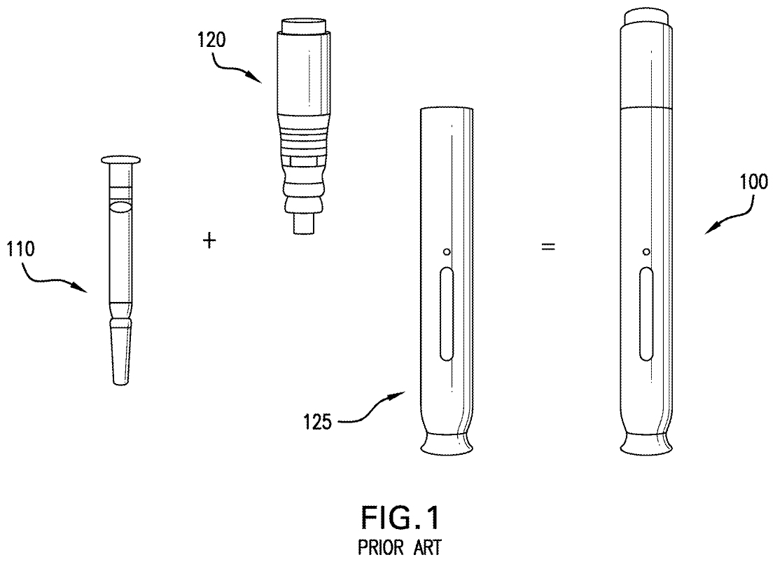

FIG. 1 is a schematic illustration of an autoinjector:

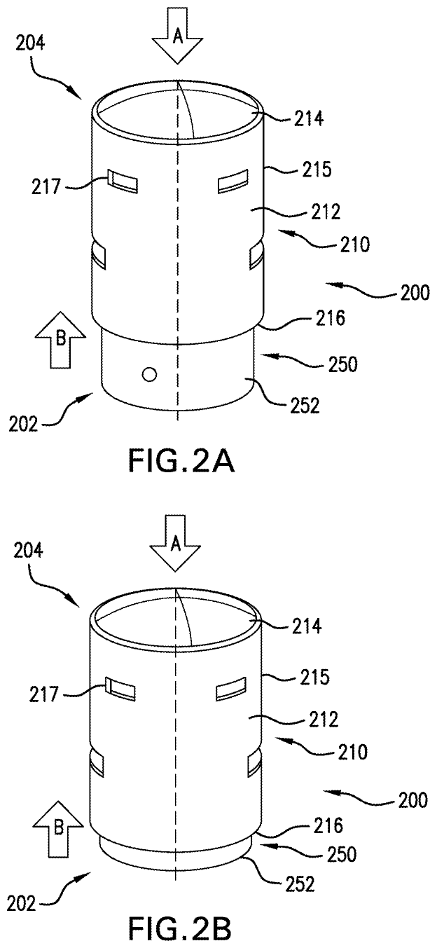

FIGS. 2A-B are schematic perspective views of a microneedle injector according to one embodiment of the present disclosure, in expanded and compressed states, respectively:

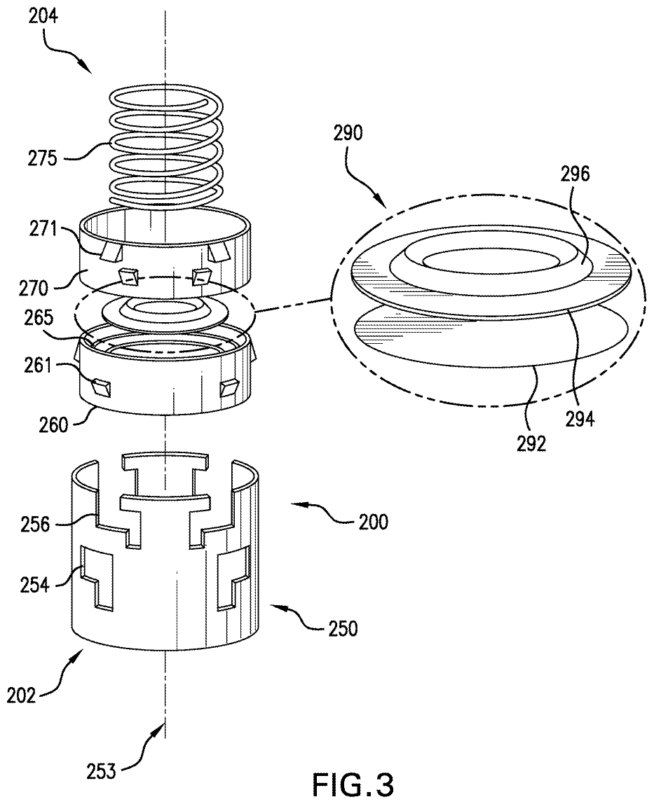

FIG. 3 is a schematic, exploded perspective view of a microneedle injector according to one embodiment of the present disclosure;

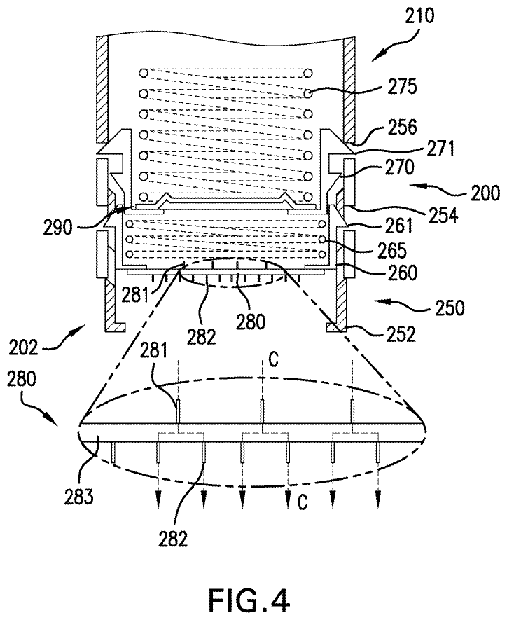

FIG. 4 is a schematic cross-sectional axial view of a microneedle injector according to one embodiment of the present disclosure:

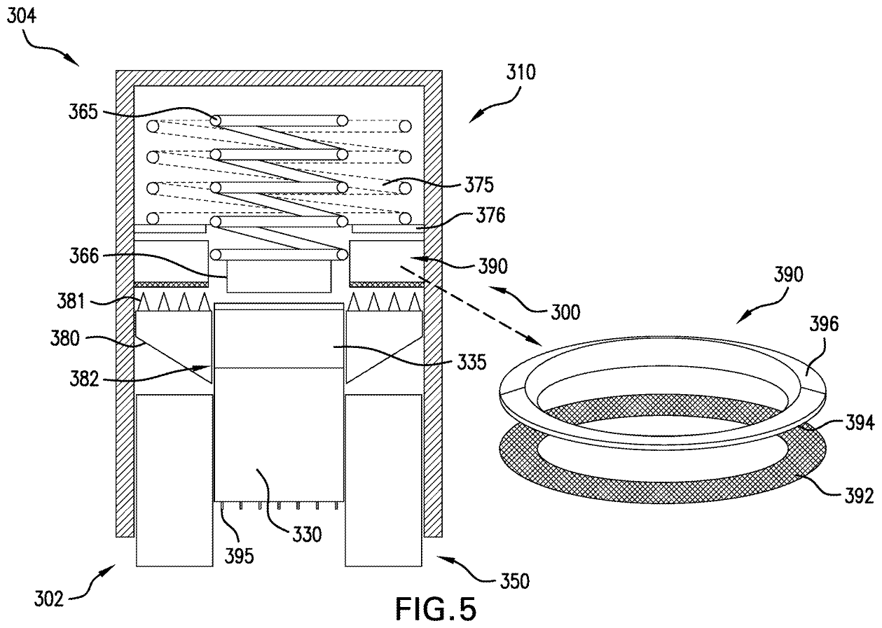

FIG. 5 is a schematic cross-sectional axial view of a microneedle injector according to another embodiment of the present disclosure; and

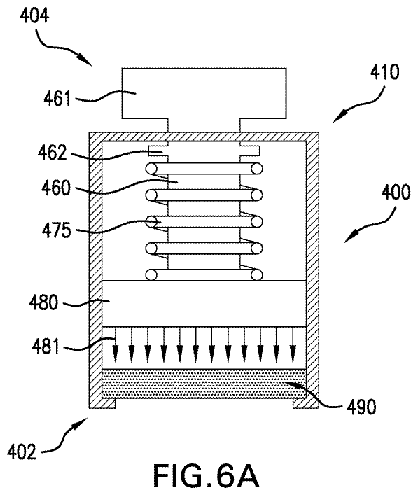

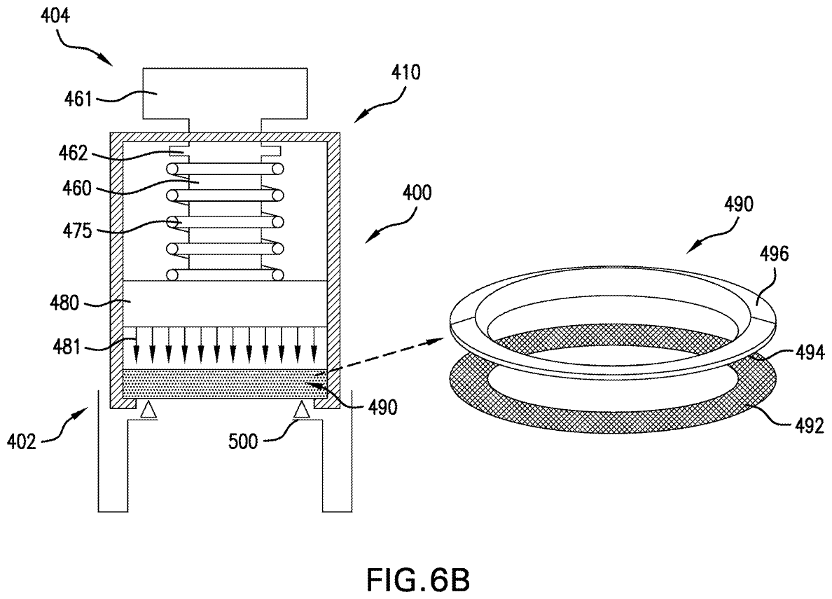

FIG. 6 is a schematic illustration of a microneedle injector according to another embodiment of the present disclosure.

Various embodiments will now be described with reference to the appended drawings. It is to be appreciated that these drawings depict only some embodiments of the disclosure and are therefore not to be considered limiting of its scope.

### DETAILED DESCRIPTION

Despite the various improvements that have been made to injectors and syringes, such as pre-filled syringes, conventional methods suffer from some shortcomings as discussed above.

Therefore, there is a need for further improvements to the devices and methods used to deliver medication. Among other advantages, the present disclosure may address one or more of these needs.

As used herein, the term “proximal,” when used in connection with a component of a syringe or injector, refers to the end of the component closest to the user's skin when using the device, whereas the term “distal,” when used in connection with a component of a syringe or injector, refers to the end of the component farthest from a needle insertion site during use.

Likewise, the terms “trailing” and “leading” are to be taken as relative to the operator's fingers (e.g., physician) of the syringe or injector. “Trailing” is to be understood as relatively close to the operator's fingers, and “leading” is to be understood as relatively farther away from the operator's fingers. Finally, as used herein, the terms “medicament,” “substance,” “drug product.” and drug are used interchangeably, and it will be understood that the injectors of the present disclosure may be used to deliver any vaccine, biologic, therapeutic, drug, drug product, chemical composition, saline, or other substance to heal or treat a disease or condition.

As shown in FIG. 1, an autoinjector 100 typically includes a traditional pre-filled syringe 110 or cartridge. The syringe 110 may be disposed between a mechanical biasing injection mechanism 120 and a shell 125. Rather than provide a medicament in a pre-filled syringe or cartridge in a typical injector, an injector having a plurality of microneedles may be used.

As shown in FIGS. 2A-B, a microneedle injector 200 may allow for transport of a drug product through a highly effective intradermal delivery mechanism. FIG. 2A illustrates the injector 200 in an expanded (or resting) state, while FIG. 2B illustrates the injector 200 in a compressed state, the injector being capable of transitioning between the expanded and the compressed states.

Injector 200 may extend between a proximal end 202 and a distal end 204 and include a generally cylindrical housing 210 adjacent distal end 204 and a generally cylindrical base 250 adjacent proximal end 202. Housing 210 may include a body 212 having a circumferential sidewall 215, a top 214, and a bottom opening 216 to receive a portion of a base. In at least some examples, housing 210 may be formed of a rigid material, such as plastics, metals, alloys, wood, or suitable combinations thereof. Housing 210 may also define a plurality of spaced windows 217 that allow the user to see inside the device to ensure that it is functioning as intended during delivery. The windows may also provide functionality as part of the design locking feature.

Base 250 may also include a generally cylindrical body 252 formed of any of the materials described above with respect to the housing 210. In at least some examples, base 250 may be slightly smaller in diameter than housing 210 and may be at least partially disposed within housing 210. In at least some examples, housing 210 and base 250) partially overlap one another to various degrees in the expanded and compressed states. Housing 210 and base 250 may be moveable relative to one another so that the two components can be actuated to transition the device to a compressed state shown in FIG. 2B by placing the base on the patient's skin and pressing top 214 toward the proximal end 202 in the direction of arrow A toward the patient's skin. Pressing top 214 will cause the injector 200 to transition to the compressed state of FIG. 2B, while inner springs will urge the device in the direction of arrow B to release the medicament and return to its expanded state after delivery of the medicament. This shield lock out feature covers the mechanism (sharps, biologics, drug) from users and provides harm protection.

Turning now to FIGS. 3-4, the inner mechanism of injector 200 will now be described. In this example, housing 210 has been removed in FIG. 3 to expose the various components inside injector 200. Moving from proximal end 202 toward the distal end, base 250 is shown, having a plurality of upper and lower cutouts 254,256 formed in body 252, the cutouts being sized to accept tabs and retainers of other components. The lower end 253 of base 250 may be open, to allow a needle unit or patch to be ejected therefrom upon activation of the device. Disposed within base 250, a generally cylindrical drug product carrier 260 may be seated within the base, the carrier 260) having a plurality of tabs 261 to be received within lower cutouts 254 and mate the two components together, the tabs 261 serving as release levers. A counter spring 265 may be seated within carrier 260 and disposed below drug product container 290. As shown in the enlarged view, drug product container 290 may be in the form of a blister pack including a lower frangible or pierceable foil seal 292 joined to a thermoformed cover 294, and a halo-shaped compartment 296 defined at least partially by thermoformed cover 294 for storing a drug product. It will be appreciated that compartment 296 may take other shapes and that dome-shaped or box-shaped compartments are also possible. Container 290 may be disposed adjacent an impactor 270, the impactor 270 having a plurality of retainers 271 to be received within upper cutouts 256 of base 250 to mate together and act as release levers. Finally, a drive spring 275 may be disposed above impactor 270 and sandwiched between the housing (not shown) and the impactor. Likewise, drug product container 290 is sandwiched between counter spring 265 and drive spring 275.

As shown in FIG. 4, injector 200 may store and eject a retractable needle unit 280 out of base 250, the activation of base 250 serving to pierce the patient's skin with microneedles of needle unit 280. One advantage of this design is that it provides a way for patients to self-administer microneedle-based therapeutics and vaccines at home and improves access to underserved markets.

The details of needle unit 280 will be appreciated with reference to the enlarged, detailed view of FIG. 4. Needle unit 280 may include one or more distal-facing piercing needles 281. In the example shown, distal-facing piercing needles 281 include a plurality of needles, and the needles may be spaced apart from one another and configured to pierce through foil seal 292 to release the contents of container 290. In at least some examples, the number, spacing and arrangement of needles 281 will complement the shape of the compartment 296 of container 290. For example, a halo-shaped compartment 296 may be used with a generally circular arrangement of piercing needles. In some embodiments, each of needles 281 may be hollow microneedles having micron-sized channels that serve as passageways for the drug product. Similarly, needle unit 280 may further include proximal-facing retractable hollow microneedles 282 disposed on a side opposite needles 281. The small, hollow microneedles 282 may penetrate the patient's skin to create micron-sized channels that the drug is directed through for delivery of vaccines, biologics, and/or therapies. In some examples, at least one of the needle unit 280 and/or container 290 may be configured to move relative to one another so that piercing needles 281 are able to pierce through the foil seal 292 and release the medicament from compartment 296. In this manner, a drug product may flow from the container 290 through needles 281, a hollow main body 283 of needle unit 280 and proximal-facing microneedles 282 through the channels shown by arrows “C”, which allow for fluid communication between the needles 281 and microneedles 282.

In some embodiments, needle unit 280 is in the form of an absorbable patch that contains drug product for delivery. In one embodiment the needle unit 280, when attached to the patient, can dissolve in, or on, the body. Alternatively, needle unit 280 may include a rigid non-absorbable material (e.g., stainless steel or plastic, etc.) with internal lumen channels, the needle unit 280 being removable after delivery.

Injector 200 may be used as an applicator which stores the needle unit 280, drug product container 290, and an optional power mechanism. In one example, when injector 200 is pressed onto the skin of a patient, base 250 will move upward, activating the device by bringing the needle unit 280 and the container 290 together. Specifically, distal-facing needles 281 of needle unit 280) may puncture the foil seal 292 of drug product container 290, filling the microneedles with the medicament. A plunger or ejector mechanism, in addition to the drive spring 275 and impactor 270, may then contact the needle unit, applying an additional load on the needle unit and ejecting it out of the base, onto the patient's skin. The plunger refers to the spring or compressive flat surface in FIG. 3.

In some embodiments the plunger mechanism will be driven by a power mechanism configured to apply a sufficient load to the microneedle array of the needle unit 280 to pierce the skin of the patient for timed release of a drug or substance. It will be understood that various actuating or power mechanisms may be used to drive the needle unit 280. In some examples, a simple compressed drive spring 275 may recoil and act as the power mechanism to drive the needle unit into the patient's skin. Alternatively, a battery-powered, gas-powered mechanism, rotary mechanism or other mechanical means may be used to drive the needle unit.

In some embodiments, device activation includes a one-step process that is activated when base 250 is pressed against the patient's skin, the pressing of base 250 being capable of compressing spring 265 and allowing needle unit 280 to pierce through container 290, and subsequently allowing drive spring 275 to be released to drive the needle unit out toward the patient's skin. In other embodiments, injector 200 may utilize a two-step process that allows the device to be initially activated when base 250 is pressed onto the patient. A second step in the process may require that the user press a safety button to trigger the device and eject the needle unit. Regardless of the mechanism used to eject the needle unit, the benefits of the microneedle injector 200 may include sustained, timed drug release and pain reduction compared to standard needles due to the use of microneedles. Additionally, injector 200 may improve access for patients, and enable at-home use.

In use, a safety cap disposed over base 250 may first be removed, the safety cap being useful for preventing the injector 200 from misfiring by preventing movement of housing 210 and base 250 relative to one another. The safety cap may also cover the bottom of base 250 to prevent contamination of the microneedles. After removing the cap, the patient (or clinician) may press base 250 against the patient's skin for activation. Alternatively, base 250 may be placed on the patient's skin, and a triggering button may be used to activate the device. Once activated (e.g., either by manual actuation or a power supply), the user may observe the inside of injector through the window of the housing to confirm movement of the needle unit 280 and/or container 290. The distal-facing needles 281 may pierce through the foil seal 292 of container 290 to release the drug product into the needle unit 280, and the needle unit may be ejected toward the patient's skin, puncturing the skin with the microneedles. The injector 200 may then be removed, and the needle unit 280 left in place with the proximal-facing microneedles piercing through the patient's skin. With the microneedles in place, the user may wait for the drug product from the needle unit to be released into the skin. After a sufficient amount of time, the needle unit may be removed. Alternatively, an absorbable or dissolvable patch 280 may be left in place. In this manner, the microneedles create a penetration site in the skin from which a blister containing drug product could be pierced and administered through the injection site created by microneedles.

In another embodiment, shown in FIG. 5, injector 300 may extend between proximal end 302 and distal end 304. In this example, injector 300 includes a housing 310 and a base 350 slidable relative to the housing. Disposed within housing 310 are a pair of springs including a counter spring 365 and a drive spring 375. In some examples, counter spring 365 may be seated in a concentric arrangement with drive spring 375 as shown. In one example, drive spring 375 terminates in a flattened compressive surface 376, and counter spring 365 terminates in a second compressive surface 366. A doughnut-shaped drug product container 390 may be seated below flattened compressive surface 375. In some examples, drug product container 390 may be in the form of a doughnut-shaped blister pack including a lower frangible or pierceable foil seal 392 joined to a thermoformed cover 394, and a halo-shaped compartment 396 defined at least partially by thermoformed cover 394 for storing a drug product similar to that described with reference to FIG. 3.

As shown in FIG. 5, injector 300 may include a fixed needle unit 380 disposed below drug product container 390, the needle unit having one or more distal-facing piercing needles 381. In the example shown, distal-facing piercing needles 381 include a plurality of needles, and the needles may be spaced apart from one another and configured to pierce through foil seal 392 to release the contents of container 390, which will flow through passages 382 of needle unit 380 into cartridge 330. As noted, cartridge 330 may be in fluid communication with the interior of needle unit 380 via passages 382 and may include a substantially linear channel that terminates in proximal-facing needles 395. A moveable stopper 335 formed of rubber or other suitable polymeric or elastomeric material may be disposed within cartridge 330, the stopper 335 being axially translatable through the cartridge 330.

In use, the operator or user may place injector 300 with base 350 adjacent the skin or tissue and push on top of housing 310 causing the housing 310 to slide toward base 350. Pressing down on housing 310 will cause drive spring 375 to compress and push compressive surface 376 to urge drug product container 390 toward needle unit 380 where the foil seal 392 will be pierced by needles 381 releasing the contents of the drug product container into needle unit 380 and out via passages 382 into cartridge 330. At the end of its travel path, drive spring 375 will release counter spring 365, which will transition from its compressed state toward its uncompressed state, this transition urging compressive surface 366 downward, pushing with it stopper 335 through cartridge 330 and forcing the fluid or contents of the drug product container through proximal-facing microneedles 395 and into the patient's tissue.

As shown in FIG. 6A, in yet another embodiment, a simple injector 400 extends between a proximal end 402 and a distal end 404 and includes a housing 410 surrounding and encasing a spring 475, a needle unit 480 having a needle matrix 481, and a drug product container 490, all similar to those described above. In this example, an axially extending plunger 460 having a thumb press 461 is provided, the plunger being moveable relative to the housing 410 and operatively coupled to the needle unit. Plunger 460 may be operatively coupled with spring 475 with, for example, laterally extending pins 462 such that actuation of the plunger downward toward the proximal end serves to compress the spring 475. In one example, spring 475 may be disposed around a portion of plunger 460. In this example, the user may place injector 400 with the proximal end being closer to the skin. The housing on the proximal end 402 is intended to create a seal to prevent the drug product from leaking out of the housing unit until the entire device is removed from the skin. The user may then press on thumb press 461 with one or more fingers to actuate the plunger 461 and release the spring. The movement of the plunger downward (against the force of spring 475) will ultimately compress the membrane 490 and push out all the drug product from the membrane. While the spring is activated, the spring may advance needle unit 480 with plunger, and specifically needles 481 through the membrane housing 490 into the patient's skin. The needles may continue through the drug product container 490 and exit the drug product container from the proximal end to deliver the medicament into the skin. In some examples, container 490 is formed of a membrane so that the needles may pierce the container without being damaged. The membrane can be formed by any non-rigid pierceable material (soft or hard polymer, plastic, etc.) that can be formed and does not have any adverse drug product compatibility issues. When the spring is fully compressed (i.e., the spring has completed its full travel), the contents (drug product) of the membrane are completely dispersed and the microneedles would have penetrated the skin. When completed, spring 475 will urge the plunger, and with it needle unit 480 upward back into the housing to reduce the risk of accidental needle sticks.

As shown in FIG. 6B, in yet another embodiment, would operate similar to FIG. 5 where an operator or user may place injector with base adjacent to the skin or tissue and push on top of housing 410 causing the housing 410 to slide toward base. When the skin sensing sleeve is compressed, it is simultaneously piercing 500 the drug product filled membrane 490. The sensing sleeve would create some seal or barrier to prevent the drug product from escaping the housing until the entire device is removed from the skin. After the skin sensing sleeve is fully depressed, the user would then activate the spring-loaded plunger rod 461. Similar to the above embodiment, once the spring is released it would drive the microneedles 481 into the patient's skin. The membrane in this embodiment is similar to FIG. 5 wherein the membrane is doughnut shaped and would allow the microneedles to bypass the membrane without piercing the membrane. The plunger 480 will ultimately compress the membrane 490 and push out all the drug product from the membrane. The drug product is creating a coating over the patient's skin and will enter the pores created by the microneedle injection sites and deliver the intended therapeutic dose. The drug product can enter the patient's skin either through the microneedle pores, or the microneedles can retract, and drug product can enter the skin through the pores.

It is to be understood that the embodiments described herein are merely illustrative of the principles and applications of the present disclosure. For example, the number, positioning and arrangement of piercing needles and microneedles of the needle unit may be varied. Additionally, the shape, dimensions and arrangement of the drug product container may be varied, and the configurations of the drug product container and the needle unit may be chosen to complement one another. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. An injector comprising:
a base:
a housing movable relative to the base:
a needle unit disposed within the base, the needle unit comprising at least one piercing needle configured to pierce a pierceable membrane of a drug product container, and at least one microneedle disposed on an opposite side of the at least one piercing needle; and
a driving mechanism configured and arranged to drive at least one of the drug product container and the needle unit toward another of the drug product container and the needle unit.

2. The injector of claim 1, further comprising a drug product container including a thermoformed cover and wherein the pierceable membrane includes a frangible foil seal.

3. The injector of claim 2, wherein the drug product container includes a halo-shaped compartment for storing a drug product.

4. The injector of claim 1, wherein the at least one piercing needle comprises a plurality of piercing needles.

5. The injector of claim 1, wherein at least one microneedle comprises a plurality of microneedles.

6. The injector of claim 1, wherein the needle unit comprises at least one channel providing fluid communication between the at least one piercing needle and the at least one microneedle.

7. The injector of claim 2, wherein the driving mechanism comprises a driving spring disposed above the drug product container and a counter spring disposed below the drug product container.

8. The injector of claim 1, wherein the base defines an open bottom through which the needle unit may pass.

9. The injector of claim 1, wherein the needle unit comprises an absorbable patch.

10. The injector of claim 1, wherein the needle unit comprises a rigid material.

11. A system for drug delivery, comprising:
a drug product container having a pierceable membrane:
a needle unit disposed adjacent the drug product container, the needle unit comprising at least one piercing needle configured to pierce the pierceable membrane of the drug product container, and at least one microneedle disposed on an opposite side of the at least one piercing needle; and
a driving mechanism configured and arranged to drive at least one of the drug product container and the needle unit toward another of the drug product container and the needle unit.

12. The system of claim 11, wherein the drug product container comprises a thermoformed cover and the pierceable membrane includes a frangible foil seal.

13. The system of claim 12, wherein the drug product container includes a halo-shaped compartment for storing a drug product.

14. The system of claim 11, wherein the at least one piercing needle comprises a plurality of piercing needles.

15. The system of claim 11, wherein at least one microneedle comprises a plurality of microneedles.

16. A method of delivering a drug product, comprising:
providing a drug product container having a pierceable membrane:
providing an injector including a housing, a base moveable relative to the housing, a needle unit disposed adjacent the drug product container, the needle unit comprising at least one piercing needle and at least one microneedle disposed on an opposite side of the at least one piercing needle, and a driving mechanism configured and arranged to drive at least one of the drug product container and the needle unit toward another of the drug product container and the needle unit.

17. The method of claim 16, further comprising the step of piercing the pierceable membrane of the drug product container with the at least one piercing needle of the injector.

18. The method of claim 16, further comprising the step of ejecting the needle unit from the base.

19. The method of claim 16, further comprising the step of piercing a patient' skin with the at least one microneedle.

20. The method of claim 16, wherein providing an injector includes providing a driving mechanism that includes at least one spring.
